(19) United States
(12) Patent Application Publication
(10) Pub. No.: US 2010/0274177 A1
(43) Pub. Date: Oct. 28, 2010

(54) Pulsed electromagnetic field and negative pressure therapy wound treatment method and system

(75) Inventors: Virginia Rybski, Marietta, GA (US); Art Loya, Prescott Valley, AZ (US); Richard A. Isenberg, Tucson, AZ (US); Stephen Soderberg, Tempe, AZ (US); Thomas Eisiminger, JR., Seattle, WA (US); Dennis Genge, Cave Creek, AZ (US)

Correspondence Address: MORRISON & FOERSTER LLP, 425 MARKET STREET, SAN FRANCISCO, CA, 94105-2482, US

(73) Assignee: REGENESIS BIOMEDICAL, INC., Scottsdale, AZ (US)

(21) Appl. No.: 12/799,370

(22) Filed: Apr. 23, 2010

Related U.S. Application Data
(60) Provisional application No. 61/214,567, filed on Apr. 24, 2009.

(51) Int. Cl. A61N 1/00; A61M 1/00

(52) U.S. Cl. 604/20

(57) ABSTRACT

A method for treating a wound of an individual and for enhancing a rate of wound healing by applying, for a first period of time, a negative pressure treatment to the wound without applying a pulsed radio frequency treatment; and applying, for a second period of time subsequent to the first period time, a pulsed radio frequency energy treatment to the wound while maintaining the negative pressure treatment to enhance the rate of wound healing. The negative pressure treatment and the pulsed radio frequency energy treatment are applied concurrently for the duration of the second period of time.

This application claims the benefit of U.S. Provisional Application No. 61/214,567, filed Apr. 24, 2009, which is hereby incorporated by reference, in its entirety.

### BACKGROUND OF THE INVENTION

1. Field

The present disclosure relates to a method of wound treatment. Specifically, the disclosure is related to a method of applying negative pressure wound treatment and pulsed radio frequency energy treatment to a wound of an individual, so as to enhance the rate of wound healing.

2. Related Art

The treatment of open wounds that are too large to spontaneously close has long been a troublesome area of medical practice. Open wounds may heal by primary intention, wherein the wound edges are brought together (apposed) and held in place by mechanical means (sutures, staples, or adhesive strips), or by secondary intention, wherein the wound is allowed to fill-in and close through the physiological wound repair process. Physiological repair of an open wound requires proliferation of subcutaneous tissue and inward migration of surrounding epithelial tissue. Some wounds, however, are sufficiently large, chronic, or infected that they are unsuitable for closure by primary intention and unable to heal spontaneously by secondary intention. In such instances, a zone of stasis in which localized edema and fibrosis restricts the flow of blood to the epithelial and subcutaneous tissue forms in the wound bed and wound periphery. Without sufficient blood flow, the wound becomes senescent, arrested in a dysfunctional disequilibrium, and/or infected; and is accordingly unable to close spontaneously. Such wounds have presented difficulties to medical personnel for many years.

A problem encountered during the treatment of wounds is the selection of an appropriate technique for wound closure during the healing process. Primary surgical closure employs sutures, adhesive strips, and/or staples to force and hold the wound edges together, allowing for rapid repair and healing. However, such devices apply a closure force to only a very small percentage of the area surrounding a wound. When there is scarring, edema, fixation, or insufficient tissue, the tension produced by the sutures can become great causing excessive pressure to be exerted by the sutures upon the tissue adjacent to each suture. As a result, the adjacent tissue often becomes ischemic thereby rendering suturing of large wounds counterproductive. If the quantity or size of the sutures is increased to reduce the tension required of any single suture, the quantity of foreign material within the wound is concomitantly increased and the wound is more apt to become infected. Additionally, the size, body location or type of a particular wound may prevent the use of sutures to promote wound closure.

One method used for treating wounds that cannot be treated by traditional means is negative pressure wound therapy. Negative pressure wound therapy has been described in U.S. Pat. No. 4,969,880 issued to Zamierowski, as well as its continuations and continuations-in-part, U.S. Pat. No. 5,100,396, U.S. Pat. No. 5,261,893, and U.S. Pat. No. 5,527,293. Further improvements and modifications of the negative pressure wound therapy are also described in U.S. Pat. No. 6,071,267, issued to Zamierowski; U.S. Pat. Nos. 5,636,643 and 5,645,081 issued to Argenta et al.; and U.S. Pat. No. 6,142,982, issued to Hunt, et al. However, one problem with negative pressure wound therapy treatment is that not all wound types respond well to the treatment.

Another method used for treating open wounds that cannot be treated by traditional means is using pulsed electromagnetic treatment devices to provide the wound with pulsed radio frequency energy. Methods for treating wound with pulsed radio frequency energy have been described in U.S. Pat. Nos. 3,043,310 and 3,181,535, issued to Milinowski; U.S. Pat. No. 3,543,762, issued to Kendall; U.S. Pat. No. 3,670,737, issued to Pearo; U.S. Pat. No. 5,584,863, issued to Rauch et al.; and U.S. Pat. No. 6,353,763, issued to George et al. However, a problem with pulsed radio frequency energy treatment is that the rate of healing can vary and some types of wounds may not respond well to the treatment.

Successful wound treatment requires an understanding of wound physiology and the mechanism of action of wound treatment therapies. With regard to wound physiology, it is known that there are three distinct phases associated with the process of wound healing. The three phases are the inflammatory phase, the proliferative phase, and the remodeling phase. During the inflammatory phase, bacteria and debris are removed and macrophages release growth factors to stimulate angiogenesis and the production of fibroblasts. Next, in the proliferative phase, granulation tissue forms and epithelialization begins, which involves migration of epithelial cells to seal the wound; fibroblasts proliferate and synthesize collagen to fill the wound and provide a strong matrix on which epithelial cells grow; and contractile cells called myofibroblasts appear in the wound and aid in wound closure. In the remodeling phase, collagen in the scar undergoes repeated degradation and resynthesis, and the tensile strength of the newly formed skin increases.

With regard to the mechanism of action of negative pressure wound therapy treatment, it is thought that the negative pressure wound therapy treatment promotes wound healing by removing excess interstitial fluid, decreasing bacterial colonization, and stimulating granulation tissue formation through micromechanical deformation. Therefore, it appears that negative pressure wound therapy treatment is effective during the inflammatory and early proliferative phases, which involve bacterial removal and granulation.

With regard to the mechanism of action of pulsed radio frequency energy treatment, it is thought that pulsed radio frequency energy treatment can stimulate growth factor production and induce cell proliferation in the wound bed. Studies have shown that pulsed radio frequency energy treatment can induce proliferation in cultured human dermal fibroblast and epithelial cells in a dose- and time-dependent fashion. Thus, it seems that pulsed radio frequency treatment is effective at propagating the proliferative and remodeling phases, which involve fibroblast and epithelial cell proliferation. Cytogenic evidence also suggests that pulsed radio frequency energy treatment modulates the inflammatory phase and stimulates angiogenesis, the stimulation of blood flow.

It would therefore be desirable to provide a method of wound treatment that enhances the rate of wound healing to wounds that do not respond well to negative pressure wound therapy treatment alone or pulsed radio frequency energy treatment alone.

Citation of the above documents, devices and studies is not intended as an admission that any of the foregoing is pertinent prior art. All statements as to the contents of these documents is based on the information available to the applicants and does not constitute any admission as to the correctness of the contents of these documents.

### BRIEF SUMMARY OF THE INVENTION

Disclosed herein is a method for treating a wound of an individual and for enhancing a rate of wound healing by applying, for a first period of time, a negative pressure treatment to the wound without applying a pulsed radio frequency treatment; and applying, for a second period of time subsequent to the first period time, a pulsed radio frequency energy treatment to the wound while maintaining the negative pressure treatment to enhance the rate of wound healing. The negative pressure treatment and the pulsed radio frequency energy treatment are applied concurrently for the duration of the second period of time.

The present disclosure also pertains to a method for treating a wound of an individual and for enhancing a rate of wound healing by applying concurrently a negative pressure treatment and a pulsed radio frequency energy treatment. The negative pressure treatment and pulsed radio frequency energy treatment of the method are maintained for a period of time sufficient to achieve the enhanced rate of wound healing. In one embodiment, the method of applying concurrently the negative pressure and pulsed radio frequency energy treatments has an enhanced rate of wound healing that results in at least a 90% decrease in wound volume.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart of one embodiment of a method for combined NPWT and PRFE wound treatment.

FIG. 2 is a flow chart of another embodiment of a method for combined NPWT and PRFE wound treatment.

FIG. 3A-3D depicts a scalp avulsion wound and the effects of healing over time with combined PRFE and NPWT treatment. (A) depicts the wound before combined treatment; (B) depicts the wound at two weeks of combined treatment; (C) depicts the wound at five weeks of combined treatment; and (D) depicts the wound at seven weeks of combined treatment.

FIG. 4 is a bar graph depicting the change in wound volume of the scalp avulsion wound over weeks of combined NPWT and PRFE treatment.

FIG. 5 is a line graph depicting the percent decrease in wound volume of the scalp avulsion wound over weeks of treatment with combined NPWT and PRFE treatment.

FIG. 6A-6D depicts a pilonidal wound healing over the course of time as a result of combined PRFE and NPWT treatment. (A) depicts the wound after 2 weeks of NPWT treatment alone; (B) depicts the wound after 1 week of combined treatment; (C) depicts the wound after 2 weeks of combined treatment; and (D) depicts the wound 2½ weeks after conclusion of combined treatment.

FIG. 7 is a bar graph depicting the change in wound volume of the pilonidal wound over weeks of combined NPWT and PRFE treatment.

FIG. 8 is a line graph depicting the percent decrease in wound volume of the pilonidal wound over weeks of combined NPWT and PRFE treatment.

FIG. 9 depicts the percent decrease in pilonidal wound area using combined NPWT and PRFE treatment compared to decreases in wound area using PRFE treatment alone and NPWT treatment alone.

FIG. 10A-10C depicts a pressure ulcer wound and the effects of healing over time with combined PRFE and NPWT treatment. (A) depicts the wound after one month of NPWT treatment; (B) depicts the wound prior to initiation of PRFE treatment; and (C) depicts the healed wound after four months of combined treatment.

FIGS. 11A and 11B depicts an Achilles tendon rupture and the effects of healing with combined PRFE and NPWT treatment, (A) depicts the wound prior to combined treatment and (B) depicts the healed wound after 78 days of combined treatment.

FIG. 12 is a line graph depicting the decrease in wound volume of the Achilles tendon rupture wound over the course of NPWT treatment and combined NPWT and PRFE treatment.

### DETAILED DESCRIPTION OF THE INVENTION

### Definitions

As used herein, negative pressure wound therapy (hereinafter “NPWT”) refers to the treatment of wounds and other damaged tissues through the application of negative pressure.

As used herein, pulsed radio frequency energy treatment (hereinafter “PRFE”) refers to the treatment of wounds and other damaged tissues through the application of pulsed, electromagnetic or magnetic energy fields oscillating at a radio frequency.

As used herein, the terms “% decrease” and “percent decrease” refer to the difference in wound volume or area before and after a given time of treatment with NPWT or PRFE alone, or in sequence, or combined NPWT and PRFE treatment. The difference in volume or area is then converted to a percentage of the original volume or area of the wound.

As used herein, the term “wound volume” refers to the dimensions of length, width, and depth of a wound of an individual. Measurement of wound volume requires measurement or approximation of wound depth, length, and width. Wound volume can be assessed manually using techniques such as filling the wound with saline, molding, or injecting dental impression material or like substance. Would volume may also be assessed digitally by using computer-assisted calibrated planimetry, structured lighting, and image processing.

As used herein, the term “wound area” refers to the dimensions of length and width of a wound of an individual. Wound area may be assessed manually by using calipers, rulers, tracings, and similar measurement devices. Wound area may also be assessed through use of computerized planimetry using digital photography and image analysis, or through ultrasound or X-ray images.

As used herein, the term “treatment for a period” refers to applying a selected treatment, or combination of treatments, at least once a day for at least 70% of days in a given period of time, where the 70% of days is rounded down. For example, treatment for a period of 2 weeks means treatment would be applied at least once a day for at least 9 days of the proscribed 2 weeks. It should be noted that the at least 70% of days may or may not be consecutive.

As used herein, the term “NPWT treatment” refers to applying negative pressure to a target wound site.

Typically, NPWT is applied either continuously or intermittently (for example, cycling on and off every few minutes) for 24-hours in a given treatment day. However, NPWT may also be applied for less than 24-hours a day. For example, even in instances in which a NPWT bandage is attached to a target wound site for an entire 24 hour period, actual negative pressure may be applied for only selected periods during the 24 hours. In a preferred treatment scenario, negative pressure is actually applied for at least 30 minutes at a time.

As used herein, the term “PRFE treatment at least once a day” refers to applying PRFE at least once a day for a period of time that ranges from at least 5 minutes to 60 minutes. For example, the length of PRFE treatment may be at least 30 minutes.

As used herein, the term “enhanced rate of wound healing” refers to a rate of wound healing achieved with combined NPWT and PRFE treatment that is greater than a rate of wound healing achieved by using only PRFE or NPWT treatment alone. Rate of wound healing is determined by measuring the decrease in wound volume or area over time. For example, rate of wound healing may be expressed as square centimeters per day or cubic centimeters per day, or as percentage of original area or volume per day, respectively. An enhanced rate of wound healing may also refer to a reduced time to wound closure, greater percentage reduction in wound area (or volume) in a given time period, or greater incidence of wound closure in a given time period.

As used herein, the term “maintaining” refers to maintaining a NPWT or PRFE treatment according to a regimen or protocol, as prescribed by a medical doctor. Accordingly, maintaining treatment takes into account that the particular prescribed regimen may include intermittent treatments. For example, a regimen for a PRFE treatment may call for two 30 minute treatments, twice daily for the duration of wound treatment. Furthermore, if the protocol calls for two 30 minute treatments twice daily and two 30 minute treatments are given on day one, skipped on the second day, and resumed on the third day, then this would still be referred to as “maintaining” the treatment regimen or protocol as long as treatment is given for at least 70% of days in a given period of time, where the 70% of days is rounded down.

As used herein, the term “concurrently” refers to the application of NPWT and PRFE treatment on a wound at the same time, taking into account that one device may be physically activated before the other, and maintaining both NPWT and PRFE therapies for a given length of time. The term “concurrently” also takes into account that that NPWT may be given at least 22 out of 24 hours per day, while PRFE may be given for 30 minutes twice daily.

As used herein in, the terms “combined treatment” and “combined NPWT and PRFE treatment” are used interchangeably and refer to concurrently using both NPWT and PRFE to treat a wound.

### Methods of Combined Wound Treatment

The following description sets forth exemplary configurations, parameters, and the like. It should be recognized, however, that such description is not intended as a limitation on the scope of the present invention, but is instead provided as a description of exemplary embodiments.

The following embodiments describe methods of combining NPWT treatment with PRFE treatment to treat an open wound that may not be closed as effectively using standard wound treatment therapies, advanced wound treatment therapies, NPWT treatment alone, or PRFE treatment alone. The combined NPWT and PRFE treatment achieves an enhanced rate of wound healing, compared to rates of wound healing achieved with either treatment alone. The method further employs prolonged, combined treatment to obtain the full benefit of the enhanced rate of wound healing.

The methods of combined NPWT and PRFE treatment described herein can be applied using any standard NPWT system that is known in the art. Briefly, NPWT systems typically include a vacuum pump, drainage tubing, and a dressing set. The pump may be stationary or portable, may rely on AC or battery power, and may allow for regulation of the negative pressure.

Certain parameters may vary between NPWT systems, for example, the negative pressure may be applied in the range of −5 to −200 mmHg, −5 to −190 mmHg, −10 to −185 mmHg, −15 to −180 mmHg, −25 to −175, −35 to −170, −45 to −165 mmHg, −50 to −160 mmHg, −60 to −150 mmHg, −70 to −125 mmHg, −75 to −115 mmHg, −85 to −110 mmHg, −90 to −100 mmHg, −91 to −99 mmHg, −92 to −97 mmHg, or −93 to −95 mmHg. In one preferred embodiment, the negative pressure is applied at −125 mmHg.

The negative pressure may also be applied continuously or intermittently, depending on the type of wound. Intermittent negative pressure may refer to, for example, a cycle of 1 minute with negative pressure on, and 1 minute with negative pressure off, a cycle of 2 minutes with negative pressure on, and 2 minutes with negative pressure off, a cycle of 3 minutes with negative pressure on, and 2 minutes with negative pressure off, a cycle of 4 minutes with negative pressure on, and 2 minute with negative pressure off, a cycle of 5 minutes with negative pressure on, and 2 minutes with negative pressure off, a cycle of 6 minutes with negative pressure on, and 2 minutes with negative pressure off, a cycle of 7 minutes with negative pressure on, and 2 minutes with negative pressure off, a cycle of 8 minutes with negative pressure on, and 2 minutes with negative pressure off, a cycle of 9 minutes with negative pressure on, and 2 minutes with negative pressure off, or a cycle of 10 minutes with negative pressure on, and 2 minutes with negative pressure off. In one embodiment, intermittent negative pressure refers to a cycle of 5 minutes with negative pressure on, and 2 minutes with negative pressure off. Moreover, negative pressure, whether applied continuously or intermittently, may be administered 24-hours a day every day for the entire period of time of wound treatment.

NPWT may be administered 24-hours a day for the entire period of time of wound treatment. NPWT may also be administered for less than 24-hours a day for the entire period of time of wound treatment. In certain embodiments, NPWT is administered for one 20-hour period, one 18-hour period one 16-hour period, one 12-hour period, one 10-hour period, one 8-hour period, two 11-hour periods, two 10-hour periods, 2 two 8-hour periods, two 6-hour periods, two 5-hour periods, two 4-hour periods, three 7-hour periods, three 6-hour periods, three 5-hour periods, three 4-hour periods, three 3-hour periods, four 5-hour periods, four 4-hour periods, four 3-hour periods, or four 2-hour periods a day for the entire period of time of wound treatment.

Additionally, the dressing sets may contain a foam, nonadherent, non-foam, woven, or moistened cotton gauze dressing to be placed in the wound and an adhesive film drape for sealing the wound. The drainage tubes may come in a variety of configurations depending on the dressings used or wound being treated.

Furthermore, any standard PRFE system known in the art can also be used for the methods described herein. Briefly, PRFE may use low-energy electromagnetic signals as a mitogenic stimulus for the treatment of wounds. PRFE systems may use a nonionizing, nonthermal radio frequency emission. The PRFE systems, for example, can have preset waveform parameters that can be regulated to ensure consistent dosing. The PRFE system may operate at a frequency of 6.78 MHz, 13.56 MHz, 27.12 MHz, 40.68 MHz, 5.8 GHz, 24.125 GHz, 61.25 GHz, 122.5 GHz, or 245.0 GHz. In one embodiment, the PRFE system operates at the Federal Communications Commission (hereinafter “FCC”) medical device frequency of 27.12 MHz, and generates an electromagnetic field that extends from the surface of the treatment applicator (antenna), allowing wounds to be treated without removal of the bandages or dressings.

The parameters of different PRFE systems may vary. For example, the electric field (E-field) strength, as measured 5 cm above the RF antenna, may range between 0.084 and 2,000 V/m, 0.1 and 1,900 V/m, 0.5 and 1,800 V/m, 1 and 1,700 V/m, 5 and 1,600 V/m, 10 and 1, 500 V/m, 25 and 1,300 V/m, 35 and 1,200 V/m, 45 and 1,000 V/m, 50 and 900 V/m, 75 and 800 V/m, 85 and 700 V/m, 90 and 600 V/m, 93 and 591 V/m, 95 and 500 V/m, 100 and 400 V/m, 150 and 400 V/m, or 200 and 350 V/m. Preferably the electric field strength, as measured 5 cm above the RF antenna is between 50 and 900 V/m. In certain embodiments, the electric field strength, as measured 5 cm above the RF antenna is 591 V/m.

The H-field strength of the PRFE system may also vary between 0.02 and 10 A/m, 0.1 and 9.5 A/m, 0.5 and 9.0 A/m, 0.75 and 8.5 A/m, 1.0 and 8.0 A/m, 1.5 and 7.5 A/m, 2.0 and 7.0 A/m, 2.5 and 6.5 A/m, 3.0 and 6.0 A/m, 3.5 and 5.5 A/m, or 4.5 and 5.0 A/m.

The E-field strength and H-field strength of the PRFE system may also be modulated individually or together. Moreover, the E-field strength and H-field strength may be optimized to treat specific wound types and soft tissue cells.

Furthermore, the radio frequency pulses of PRFE systems may range between 16 and 3000 microsecond pulses, 18 and 1500 microsecond pulses, 20 and 750 microsecond pulses, 22 and 500 microsecond pulses, 24 and 250 microsecond pulses, 26 and 125 microsecond pulses, 28 and 75 microsecond pulses, 30 and 65 microsecond pulses, 30 and 55 microsecond pulses, 30 and 45 microsecond pulses, 30 and 42 microsecond pulses, or 30 and 35 microsecond pulses. Preferably the radio frequency pulses range between 30 and 65 microsecond pulses. More preferably the radio frequency pulses range between 30 and 45 microsecond pulses. In one embodiment, the radio frequency pulses at 42 microsecond pulses.

Additionally, the pulse frequency of PRFE systems may range between land 1000 pulses per second, 25 and 900 pulses per second, 50 and 800 pulses per second, 100 and 700 pulses per second, 200 and 600 pulses per second, or 300 and 500 pulses per second. In a preferred embodiment, the pulse frequency is 1000 pulses per second.

The pulse interval of PRFE systems may also vary between land 800 microseconds, 5 and 600 microseconds, 10 and 500 microseconds, 15 and 400 microseconds, 20 and 200 microseconds, 25 and 100 microseconds, 50 and 75 microseconds, or 55 and 65 microseconds.

Moreover, the duty cycle of PRFE systems may range between 0.4% and 10%, 0.6% and 9.5%, 0.8% and 9.0%, 1.0% and 8.5%, 1.5% and 8.0%, 2.0% and 7.5%, 2.5% and 7.0%, 3.0% and 6.5%, 3.5% and 6.0%, 4.0% and 5.5%, or 4.5% and 5.0%.

In one embodiment, the PRFE system is the Provant® Therapy System, available from Regenesis® Biomedical of Scottsdale, Ariz.

The methods of combined NPWT and PRFE treatment described herein can utilize any of the NPWT and PRFE treatments systems and protocols described herein or otherwise known in the art.

Typically, initiation of NPWT treatment involves placing a shaped wound cover, operably connected to a vacuum pump, substantially over a wound of an individual. The shaped wound cover would define a covered volume above the wound, and the covered volume would have a gas pressure at an initial pressure. After placing the shaped wound cover substantially over the wound, negative pressure would be applied by activating the vacuum pump. The vacuum pump would lower the gas pressure of the covered volume from the initial pressure to a reduced pressure. Typically, initiation of PRFE treatment involves placing over the wound a treatment applicator that is configured to deliver the pulsed radio frequency energy. The treatment applicator would be connected to a pulsed radio frequency signal generator. Once the treatment applicator has been placed over the wound, the generator would deliver the pulsed radio frequency signal to the applicator, and the applicator would deliver the pulsed radio frequency energy to the wound. In one exemplary method of combined treatment, the PRFE applicator may be placed directly over a NPWT shaped wound cover.

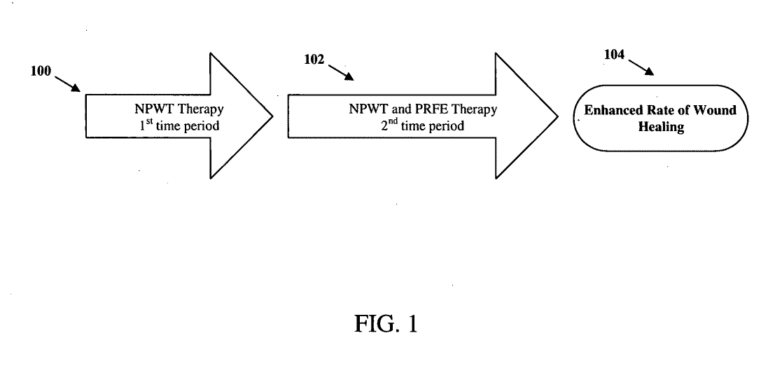

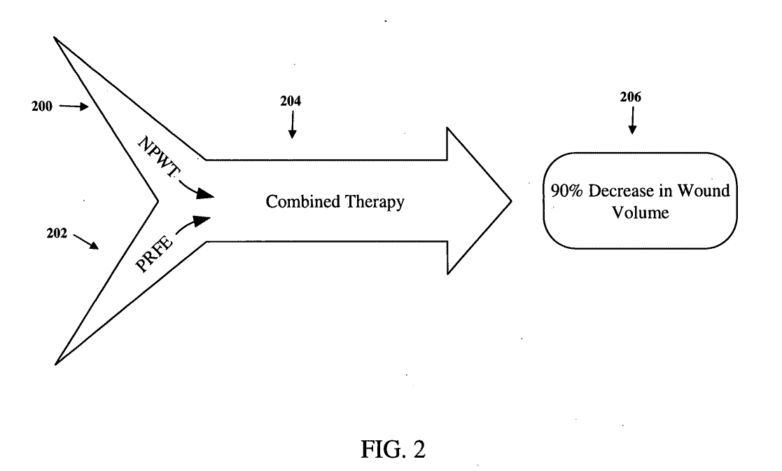

Referring now to the drawings, where like elements are designated by like reference numerals throughout, FIG. 1 and FIG. 2 depict methods of combined treatment comprising applying NPWT and PRFE to a wound of an individual to achieve an enhanced rate of wound healing.

### NPWT Pre-Treatment Prior to Combined Treatment

In one embodiment, shown in FIG. 1, a method of combined NPWT and PRFE treatment for treating a wound of an individual involves first pre-treating the wound with a NPWT treatment for a first period of time 100. Preferably the NPWT treatment is given in the absence of a PRFE treatment during the first period of time 100. After the end of the NPWT pre-treatment period of time 100, the wound is treated for a second period of time with a combined treatment of NPWT and PRFE 102. Preferably, the combined treatment is initiated immediately after the end of the NPWT pre-treatment period of time 100. However, a delay between the period of pre-treatment and combined treatment is acceptable. The NPWT and PRFE treatments are applied concurrently for the duration of the second period of time 102. The combined treatment is maintained for a second period of time 102 that is sufficient to achieve an enhanced rate of wound healing 104.

In certain embodiments the NPWT treatment is applied intermittently to the wound. In other embodiments the NPWT treatment is applied continuously.

In other embodiments, NPWT may be administered 24-hours a day for the entire period of time of wound treatment. In still other embodiments, NPWT may also be administered for less than 24-hours a day for the entire period of time of wound treatment. In certain embodiments, NPWT is administered for one 20-hour period, one 18-hour period one 16-hour period, one 12-hour period, one 10-hour period, one 8-hour period, two 11-hour periods, two 10-hour periods, 2 two 8-hour periods, two 6-hour periods, two 5-hour periods, two 4-hour periods, three 7-hour periods, three 6-hour periods, three 5-hour periods, three 4-hour periods, three 3-hour periods, four 5-hour periods, four 4-hour periods, four 3-hour periods, or four 2-hour periods a day for the entire period of time of wound treatment.

In yet another embodiment, the NPWT treatment is applied during the interval between the first period of time 100 and the second period of time 102. Alternatively, in some embodiments the NPWT treatment is discontinued at the end of the first period of time 100, and is initiated and maintained again throughout the second period of time 102.

In another embodiment, the PRFE treatment is applied at least once a day, twice a day, three times a day, four times a day, five times a day, six times a day, seven times a day eight times a day, nine times a day, ten times a day, or more for a period of time that ranges from at least 5 minutes to 60 minutes, 5 minutes to 55 minutes, 5 minutes to 50 minutes, 5 minutes to 45 minutes, 5 minutes to 40 minutes, 5 minutes to 35 minutes, 5 minutes to 30 minutes, 5 minutes to 25 minutes, 5 minutes to 20 minutes, 5 minutes to 15 minutes, or 5 minutes to 10 minutes. In certain embodiments, the period of time is 50 minutes, 45 minutes, 40 minutes, 35 minutes, 30 minutes, 25 minutes, 20 minutes, 15 minutes, 10 minutes, or 5 minutes.

In further embodiments, the length of the first period of time 100 may vary. For example, the first period of time 100 may be at least one day, two days, three days, four days, five days, six days, one week, one and a half weeks, two weeks, two and half weeks, three weeks, three and a half weeks, four weeks, four and half weeks, five weeks, five and a half weeks, six weeks, six and a half weeks, seven weeks, seven and a half weeks, or two months. In a certain embodiment, the first period of time 100 is at least two weeks. In another embodiment, the first period of time 100 is at least one week.

The length of the second period of time 102 may also vary. For example, the second period of time 102 may be at least one day, two days, three days, four days, five days, six days, one week, one and a half weeks, two weeks, two and half weeks, three weeks, three and a half weeks, four weeks, four and half weeks, five weeks, five and a half weeks, six weeks, six and a half weeks, seven weeks, seven and a half weeks, two months, two and half months, three months, three and a half months, four months, four and a half months, five months, five and a half months, six months, or longer. In a preferred embodiment, the length of the second period of time 102 is at least one week.

The enhanced rate of wound healing 104 may result in a in wound volume or wound area that is, for example, at least 5%, 10%, 15%, 20%, 25%, 30%, 35%, 40%, 45%, 50%, 55%, 60%, 65%, 70%, 75%, 80%, 85%, 90%, 95%, or 99% smaller than that achieved by either NPWT or PRFE treatment alone. In a certain embodiment, the enhanced rate of wound healing 104 results in a wound volume or wound area that is at least 25% smaller than that achieved by either NPWT or PRFE treatment alone.

The percentage change in wound volume or wound area may be calculated by taking the difference in percentage between the combined NPWT and PRFE treatment over a given period of time and the NPWT or PRFE treatment alone over the same period of time. For example, if the decrease in wound volume using the combined treatment was 85% and the decrease in wound volume using NPWT treatment alone was 60%, then the difference in percentage would be 25%.

In certain embodiments, the enhanced rate of wound healing 104 may result in, for example, at least a 10%, 15%, 20%, 25%, 30%, 35%, 40%, 45%, 50%, 55%, 60%, 65%, 70%, 75%, 80%, 85%, 90%, 95%, 99%, or 100% decrease in wound volume or wound area, over the total treatment period of time 100 and 102. In a preferred embodiment, the enhanced rate of wound healing 104 results in at least a 90% decrease in wound volume, over the total treatment period of time 100 and 102.

The enhanced rate of wound healing may result in a wound volume or wound area that decreases at a rate of at least 1%/week, 1.5%/week, 2%/week, 3%/week, 4%/week, 5%/week, 6%/week, 7%/week, 8%/week, 9%/week, 10%/week, 15%/week, 20%/week, 25%/week, 30%/week, 35%/week, 40%/week, 45%/week, 50%/week, 55%/week, 60%/week, 65%/week, 70%/week, 75%/week, 80%/week, 85%/week, 90%/week, 95%/week, or 100%/week.

The enhanced rate of wound healing may also result in a wound volume that decreases at a rate of at least 1 cm³/week, 5 cm³/week, 10 cm³/week, 15 cm³/week, 20 cm³/week, 25 cm³/week, 30 cm³/week, 35 cm³/week, 40 cm³/week, 42 cm³/week, 45 cm³/week, 50 cm³/week, 60 cm³/week, 70 cm³/week, 75 cm³/week, 80 cm³/week, 90 cm³/week, 95 cm³/week, or 100 cm³/week. In one embodiment, the enhanced rate of wound healing results in a wound volume that decreases at a rate of at least 42 cm³/week

The enhanced rate of wound healing may further result in a wound area that decreases at a rate of at least 1 cm²/week, 5 cm²/week, 10 cm²/week, 15 cm²/week, 20 cm²/week, 25 cm²/week, 30 cm²/week, 35 cm²/week, 40 cm²/week, 42 cm²/week, 45 cm²/week, 50 cm²/week, 60 cm²/week, 70 cm²/week, 75 cm²/week, 80 cm²/week, 90 cm²/week, 95 cm²/week, or 100 cm²/week. In one embodiment, the enhanced rate of wound healing results in a wound volume that decreases at a rate of at least 42 cm²/week.

The method of combined treatment for treating a wound described in FIG. 1 may be used to treat various types of wounds. For example, the combined treatment may be used to treat: chronic wounds; large, deep, open wounds; graft and flap site wounds; full thickness burns; partial thickness burns; diabetic ulcers; pressure ulcers; decubitus ulcers; arterial ulcers; avulsion injuries; pilonidal disease; cysts; acute wounds; tendon rupture wounds; postoperative incisions; postoperative wounds; traumatic wounds; dermatology conditions; scleroderma; atrophy blanche disease; trauma; bomb blast or other military-type inflicted wounds; gunshot wounds; bites; or wound dehiscence. It should be understood that the method of combined NPWT and PRFE treatment may be used to treat one or more wounds of an individual. It should be understood that the method of combined NPWT and PRFE treatment may be used to concurrently or sequentially treat one or more wounds of an individual.

### Prolonged Combined Treatment

In another embodiment, depicted in FIG. 2, a method of combined treatment 204 for treating a wound of an individual involves applying concurrently NPWT 200 and PRFE 202 treatments to the wound. The combined treatment 204 results in an enhanced rate of wound healing. The combined treatment 204 may be maintained for a period of time sufficient to achieve at least 100%, 99%, 98%, 95%, 90%, 85%, 80%, 75%, 70%, 65%, 60%, 55%, 50%, 45%, 40%, 35%, 30%, 25%, 20%, 15%, 13%, 10%, decrease in wound volume 206 or in wound area. Preferably, the combined treatment 204 is maintained for a period of time sufficient to achieve at least a 90% decrease in wound volume 206 or wound area.

In one embodiment resulting in at least a 90% decrease in wound volume, the combined treatment 204 is maintained for 3 weeks. In other embodiments, the period of time that the combined treatment 204 is maintained may vary, for example, it may be at least one day, two days, three days, four days, five days, six days, one week, one and a half weeks, two weeks, two and half weeks, three weeks, three and a half weeks, four weeks, four and half weeks, five weeks, five and a half weeks, six weeks, six and a half weeks, seven weeks, seven and a half weeks, two months, two and half months, three months, three and a half months, four months, four and a half months, five months, five and a half months, six months, or longer.

The enhanced rate of wound healing may result in a in wound volume or wound area that is, for example, at least 5%, 10%, 15%, 20%, 25%, 30%, 35%, 40%, 45%, 50%, 55%, 60%, 65%, 70%, 75%, 80%, 85%, 90%, 95%, or 99% smaller than that achieved by either NPWT or PRFE treatment alone. In a preferred embodiment, the enhanced rate of wound healing results in a wound volume or area that is at least 25% smaller than that achieved by either NPWT or PRFE treatment alone.

The enhanced rate of wound healing may also result in a wound volume or wound area that decreases at a rate of at least 1%/week, 1.5%/week, 2%/week, 3%/week, 4%/week, 5%/week, 6%/week, 7%/week, 8%/week, 9%/week, 10%/week, 15%/week, 20%/week, 25%/week, 30%/week, 35%/week, 40%/week, 45%/week, 50%/week, 55%/week, 60%/week, 65%/week, 70%/week, 75%/week, 80%/week, 85%/week, 90%/week, 95%/week, or 100%/week.

The enhanced rate of wound healing may further result in a wound volume that decreases at a rate of at least 1 cm³/week, 5 cm³/week, 10 cm³/week, 15 cm³/week, 20 cm³/week, 25 cm³/week, 30 cm³/week, 35 cm³/week, 40 cm³/week, 42 cm³/week, 45 cm³/week, 50 cm³/week, 60 cm³/week, 70 cm³/week, 75 cm³/week, 80 cm³/week, 90 cm³/week, 95 cm³/week, or 100 cm³/week. In one embodiment, the enhanced rate of wound healing results in a wound volume that decreases at a rate of at least 42 cm³/week.

The enhanced rate of wound healing may also result in a wound area that decreases at a rate of at least 1 cm²/week, 5 cm²/week, 10 cm²/week, 15 cm²/week, 20 cm²/week, 25 cm²/week, 30 cm²/week, 35 cm²/week, 40 cm²/week, 42 cm²/week, 45 cm²/week, 50 cm²/week, 60 cm²/week, 70 cm²/week, 75 cm²/week, 80 cm²/week, 90 cm²/week, 95 cm²/week, or 100 cm²/week. In one embodiment, the enhanced rate of wound healing results in a wound volume that decreases at a rate of at least 42 cm²/week.

In certain embodiments, the NPWT treatment is applied intermittently to the wound. In other embodiments the NPWT treatment is applied continuously.

In other embodiments, NPWT is administered 24-hours a day for the entire period of time of wound treatment. NPWT may also be administered for less than 24-hours a day for the entire period of time of wound treatment. In still other embodiments, NPWT is administered for one 20-hour period, one 18-hour period one 16-hour period, one 12-hour period, one 10-hour period, one 8-hour period, two 11-hour periods, two 10-hour periods, 2 two 8-hour periods, two 6-hour periods, two 5-hour periods, two 4-hour periods, three 7-hour periods, three 6-hour periods, three 5-hour periods, three 4-hour periods, three 3-hour periods, four 5-hour periods, four 4-hour periods, four 3-hour periods, or four 2-hour periods a day for the entire period of time of wound treatment.

In a further embodiment, the PRFE treatment is applied at least once a day, twice a day, three times a day, four times a day, five times a day, six times a day, seven times a day eight times a day, nine times a day, ten times a day, or more for a period of time that ranges from at least 5 minutes to 60 minutes, 5 minutes to 55 minutes, 5 minutes to 50 minutes, 5 minutes to 45 minutes, 5 minutes to 40 minutes, 5 minutes to 35 minutes, 5 minutes to 30 minutes, 5 minutes to 25 minutes, 5 minutes to 20 minutes, 5 minutes to 15 minutes, or 5 minutes to 10 minutes. In certain embodiments, the period of time is 50 minutes, 45 minutes, 40 minutes, 35 minutes, 30 minutes, 25 minutes, 20 minutes, 15 minutes, 10 minutes, or 5 minutes.

The method of combined treatment for treating a wound described in FIG. 2 may be used to treat various types of wounds. For example, the combined treatment may be used to treat: chronic wounds; large, deep, open wounds; graft and flap site wounds; full thickness burns; partial thickness burns; diabetic ulcers; pressure ulcers; decubitus ulcers; arterial ulcers; avulsion injuries; pilonidal disease; cysts; acute wounds; tendon rupture wounds; postoperative incisions; postoperative wounds; traumatic wounds; dermatology conditions; scleroderma; atrophy blanche disease; trauma; bomb blast or other military-type inflicted wounds; gunshot wounds; bites; or wound dehiscence. It should be understood that the method of combined NPWT and PRFE treatment may be used to treat one or more wounds of an individual.

### EXAMPLES

### Example 1

### Treatment and Closure of an Avulsed Scalp Wound with Significant Bone Exposure Using Pulsed Radio Frequency Energy Treatment and Negative Pressure Treatment

### Background

Wounds involving exposed bone are categorically difficult to manage and slow to heal. Historically, traumatic scalp avulsions have been treated with complex musculo-cutaneous flaps, skin grafts², or pure secondary intention³. Successful modern treatment of these wounds demands aggressive, comprehensive combination therapies to expedite granulation, contraction and epithelialization. This example describes the use of a pulsed radio frequency energy treatment (PRFE)^{4,5} in conjunction with negative pressure wound therapy (NPWT) in the treatment of a serious scalp avulsion.

### Case Report

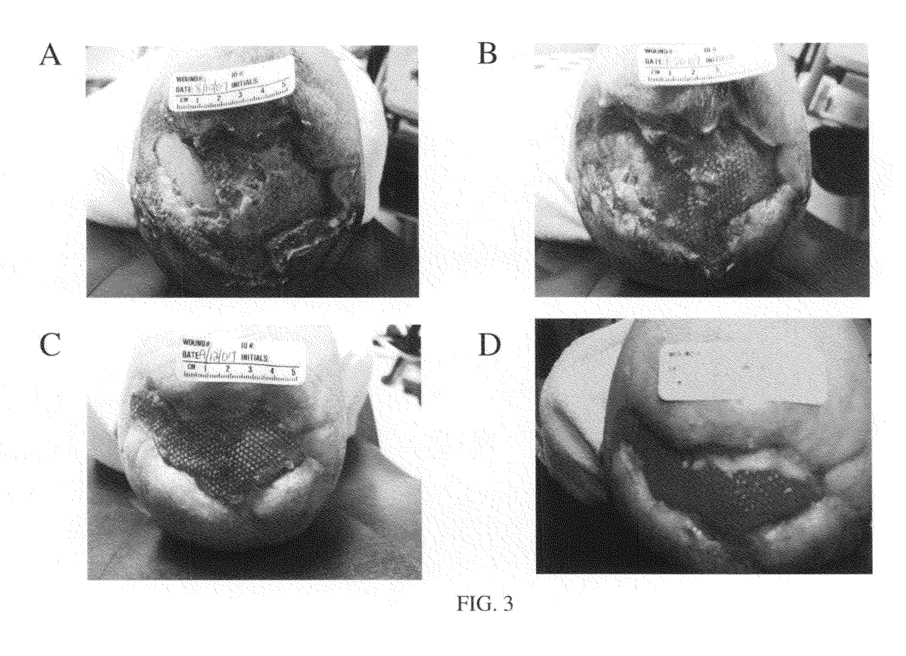

A 63 year old female presented to the hospital following a roll-over motor vehicle accident. On examination, a full-thickness avulsion injury was identified, with virtually complete detachment of the skin, muscle and fascia on the crown of the head. Despite the type and extent of trauma, the patient had no cognitive deficits. Her co-morbidities included diabetes, hypertension and hyperlipidemia. The patient was indigent and illiterate. An initial attempt at surgical closure with approximation of the skin flap failed and the necrotic flap was surgically debrided one week later. The resulting wound was 18 cm×12 cm with exposed skull (FIG. 3A). NPWT treatment was initiated during hospitalization using a standard protocol that included applying continuous pressure at −125 mmHg for the length of the treatment; and continued as an outpatient. NPWT systems and protocols are well known in the art. For example, the V.A.C.® (KCI) system is described in, “V.A.C.® Therapy Clinical Guidelines: A reference source for clinicians,” Kinetics Concepts Inc. (KCI), July, 2007.

### Methods

The NPWT treatment, using the ActiV.A.C.® (KCI) system, was performed by applying to the wound an open-celled reticulated foam dressing that sealed the wound to maintain a vacuum. Specifically, the open pore white polyvinyl alcohol foam (V.A.C.® WhiteFoam Dressing) dressing was cut to fit the portions of the wound bed with exposed bone, and the black open pore reticulated polyurethane foam (V.A.C.®GranuFoam®) was cut to fit the portions of the wound bed without exposed bone. The foam was placed into the wound bed and held in place with a transparent adhesive drape. Once the dressing was applied, an evacuation tube ran from the wound through the dressing, drawing excess exudates away from the wound and depositing them into a canister attached at the other end. The canister was attached to a vacuum pump that provided continuous negative pressure for the duration of the treatment. Pressure was applied at −125 mmHg. The foam dressings were changed every Monday, Wednesday, and Friday.

After one week of outpatient NPWT treatment, a combined treatment was initiated by adding PRFE treatment (Provant® Therapy System, Regenesis Biomedical Inc., Scottsdale Ariz.) to the NWPT treatment. Treatment was performed twice daily for 30 minutes. PRFE treatment was delivered through a solid-state 27.12 MHz fixed power output radiofrequency generator (Provant® Therapy System, Regenesis Biomedical, Inc., Scottsdale, Ariz.), which transmits a fixed dose of nonionizing, nonthermal radiofrequency energy, at an electric field strength of 591 V/m, and with 42 microsecond pulses delivered at 1000 pulses per second, into the wound bed to promote healing. The PRFE was applied through intact NPWT foam dressings and apparatus. The patient was treated at home with weekly wound clinic visits.

### Results

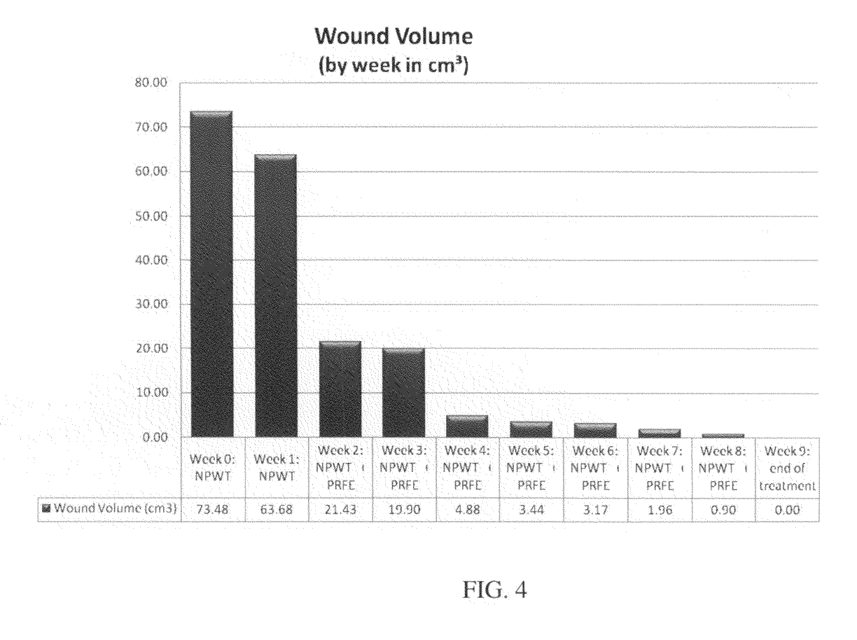

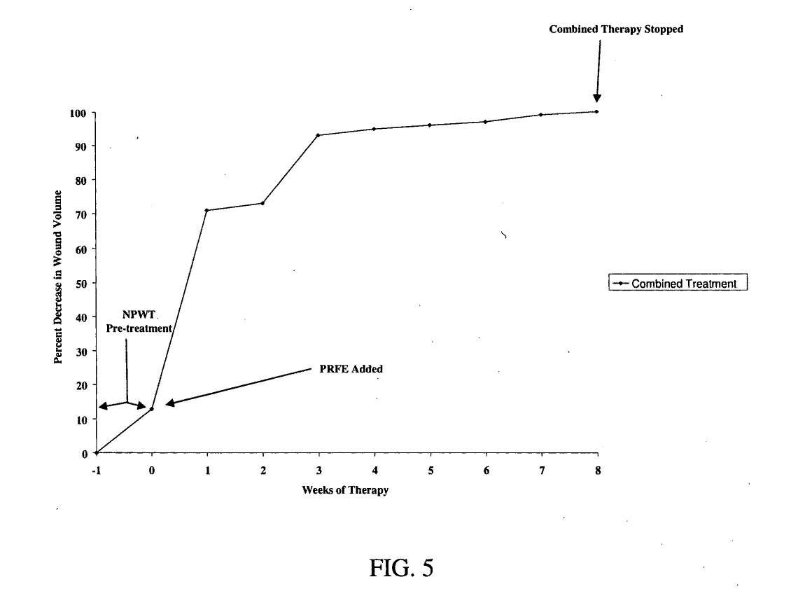

Following debridement of the necrotic skin flap, the wound volume measured 73.48 cm³ (FIG. 3A and FIG. 4). A pre-treatment with NPWT treatment alone was conducted for one week (FIG. 4 and FIG. 5). After the NPWT pre-treatment, the wound volume decreased by 13% to 63.68 cm³ (TABLE 1 and FIG. 4). After the one week of NPWT pre-treatment, the combined treatment was initiated by adding PRFE treatment to the NPWT treatment. One week of combined treatment resulted in a 71% decrease in wound volume (FIG. 4 and FIG. 5). With the combined NPWT and PRFFE treatment, the wound had decreased in volume by 93% after three weeks of treatment (FIG. 4 and FIG. 5). Maintaining the combined treatment lead to closure of the wound by week 9 of treatment (FIG. 3D and TABLE 1). The patient tolerated the combination of NPWT and PRFE well and experienced no complications or adverse events.

TABLE 1

| Week of | Wound | Percent | |
|---|---|---|---|
| Treatment | Volume | Decrease | Therapies Used |
| 0 | 73.48 cm³ | 0 | Initiation of NPWT alone |
| 1 | 63.68 cm³ | 13% | PRFE Added |
| 2 | 21.43 cm³ | 71% | Combined Treatment |
| 3 | 19.90 cm³ | 73% | Combined Treatment |
| 4 | 4.88 cm³ | 93% | Combined Treatment |
| 5 | 3.44 cm³ | 95% | Combined Treatment |
| 6 | 3.17 cm³ | 96% | Combined Treatment |
| 7 | 1.96 cm³ | 97% | Combined Treatment |
| 8 | 0.90 cm³ | 99% | Both therapies stopped |
| 9 | 0 cm³ | 100% | Discharged |

### Conclusions

Combined NPWT and PRFE treatment, in conjunction with a NPWT pre-treatment, was found to enhance the rate of wound closure in this patient with extensive, severe scalp injuries. The NPWT pre-treatment gave a healing rate of about 1.4 cm³/week while the combined NPWT and PRFE treatment lead to a healing rate of about 42 cm³/week (TABLE 1).

Despite the extensive surface of exposed bone, the wound granulated and closed rapidly (FIG. 3D). Additional surgical closure of wound using skin flaps was avoided.

### Example 2

### Treatment of Pilonidal Wound Using Pulsed Radio Frequency Energy and Negative Pressure Wound Therapy

### Introduction

Pilonidal (herein “PN”) disease occurs commonly among young men (incidence is 1.1% among male college students) and generates considerable morbidity and disability, including chronic sacral wounds, loss of productivity and lifestyle limitation⁶. Risk factors include Caucasian race, increased sweating associated with sitting and buttock friction, poor personal hygiene, obesity and local trauma. While generally considered an acquired disease, some authorities assert that PN disease is congenital⁷.

Among military personnel, PN has historically been a leading cause of nontraumatic sick days. The literature cites a recovery time approximating 100 days^{8,9}. 80,000 US Army soldiers were hospitalized with pilonidal sinus disease for an average of 55 days during World War II¹⁰. During one year of the Vietnam conflict, 2,075 US Navy sailors required 90,392 sick days for treatment of the condition¹¹. An unfortunate and common sequel of PN surgery has been chronic, non-healing wounds¹².

In a retrospective study of 141 PN patients by C Perruchoud (2002), treatment with excision and open granulation led to an average length of hospital stay of 4 days, 40 post-operative visits, 38 days lost from work, and a time to complete healing of 72 days (10.2 weeks)¹³. In another study, negative pressure wound therapy (NPWT) treatment was added to the treatment protocol with a mean time to complete epithelialization of 12 weeks¹⁴. While the time to healing is not markedly different in these two studies, NPWT treatment has become widely used as an adjunctive treatment for pilonidal disease¹⁵. In vitro studies have suggested that this technology may be beneficial in the repair of chronic wounds^{16,17.}

Reported herein is the case of a young man whose pilonidal cyst was treated with surgical debridement and primary closure, followed by dehiscence and attempted healing by secondary intention. The wound failed to respond to conventional treatment and NPWT alone, but ultimately responded briskly to a combined NPWT and PRFE treatment.

### Methods

The NPWT treatment, using the ActiV.A.C.® (KCI) system, was performed by applying to the wound the black open-celled reticulated GranuFoam® dressing, covered by the transparent adhesive drape that sealed the wound to maintain a vacuum. Once the dressing was applied, an evacuation tube ran from the wound through the dressing, drawing excess exudates away from the wound and into a canister attached at the other end. The canister was attached to a vacuum pump that provided negative pressure according to standard V.A.C.® (KCI) protocol of applying continuous pressure at −125 mmHg for the duration of the treatment. The foam dressings were changed every two to three days by skilled nursing personnel.

PRFE treatment was delivered through a solid-state 27.12 MHz fixed power output radiofrequency generator (Provant® Therapy System, Regenesis Biomedical, Inc., Scottsdale, Ariz.), which transmits a fixed dose of nonionizing, nonthermal radiofrequency energy, at an electric field strength of 591 V/m, and with 42 microsecond pulses delivered at 1000 pulses per second, into the wound bed to promote healing. The PRFE treatment was applied through intact NPWT foam dressings and apparatus.

### Case History and Results

A 15 year old boy presented to the Naval Hospital Bremerton outside of Seattle, Wash. with the chief complaint of foul smelling drainage from his post-sacral area for four months. The patient's height and weight were 6′2″ and 240 pounds. He had no relevant medical history and no systemic symptoms. He had extensive hair growth on his back and buttocks. A large sinus opening with protruding clumps of hair and draining pus was found in the midline of the post-sacrococcygeal area and diagnosed as a post sacral pilonidal cyst.

Gross surgical dissection was performed and a tissue mass measuring 8×5×4.5 cm was submitted to pathology. The wound was closed primarily and drain tubes placed. The edge of the dermis was sutured to the post sacral fascia. The resulting wound was 1 cm wide. Pathology revealed a pilonidal cyst and secondary finding of adjacent atypical compound nevus. The post-op regimen included showers with wound cleanser twice daily and after bowel movements. Rolled gauze was placed in the wound bed and changed every 2-4 hours. The patient was compliant with these instructions. At one week post-op, he was afebrile with mild discomfort at the operative site. Fecal debris was found in the wound bed. More aggressive cleansing and frequent dressing changes were implemented (one roll of gauze between his gluteal clefts every two hours).

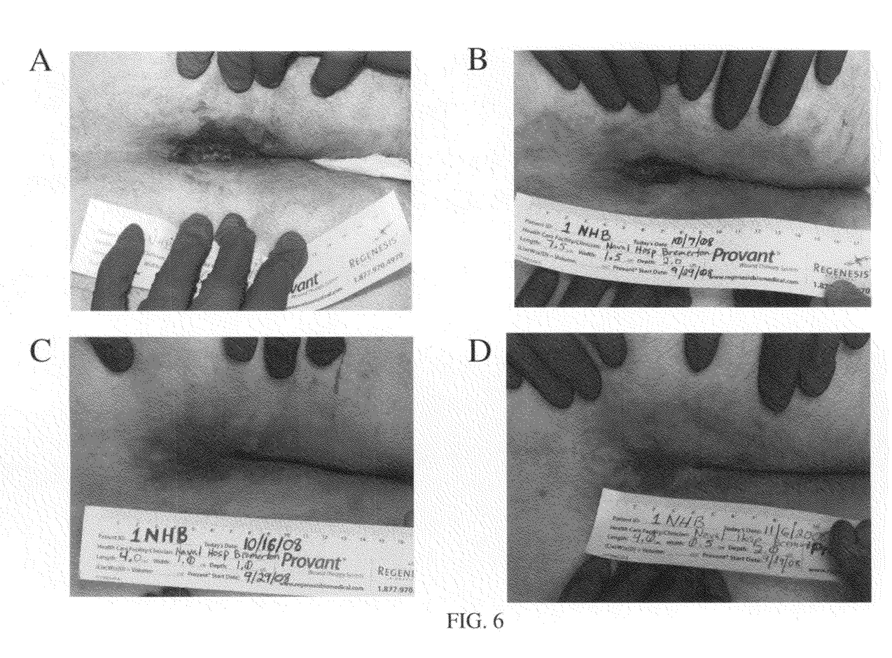

Two weeks after the initial surgery the patient presented with a partial dehiscence of the wound with wound edge necrosis. The wound was cleaned and revised a second time, and left open to heal secondarily. The resultant wound measured 10×2×4 cm (volume 80.0 cm³). Due to the depth and width of the wound, NPWT treatment was implemented in order to reduce maceration and encourage granulation. At the end of two weeks of NPWT treatment, the wound was clean and free of infection but no granulation tissue or reduction in dimension or volume was noted (FIG. 6A).

Because of the failure to respond to conventional and NPWT treatment, PRFE treatment was added to the NPWT treatment. Pursuant to the PRFE treatment protocol, the patient was placed in a comfortable position, with the PRFE applicator pad placed directly adjacent to the patient's dressed wound on the gluteal cleft. PRFE treatment was administered twice daily for 30 minutes with good compliance. All PRFE treatments were performed at home without skilled nursing supervision. The NPWT dressings were left in place during PRFE treatments.

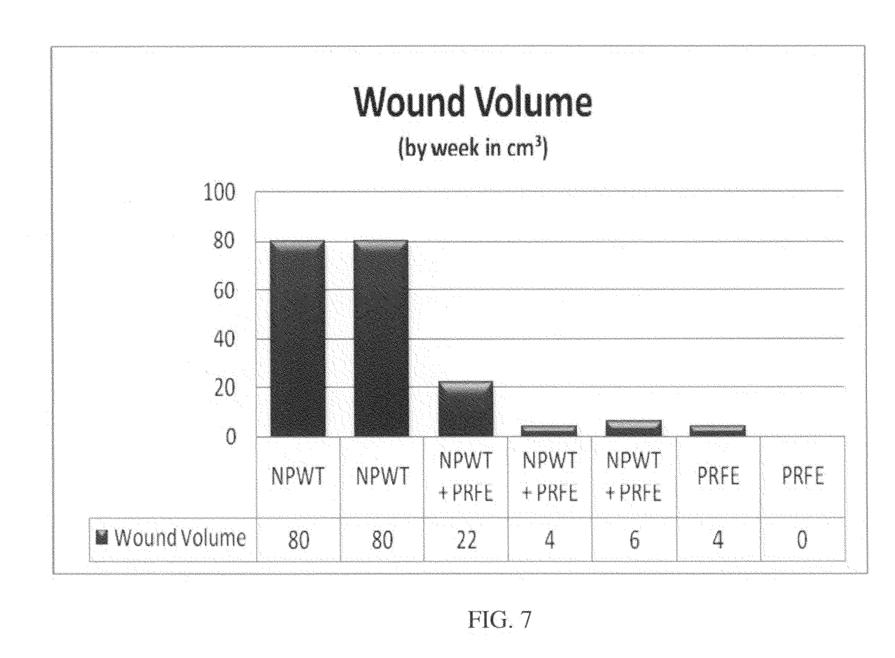

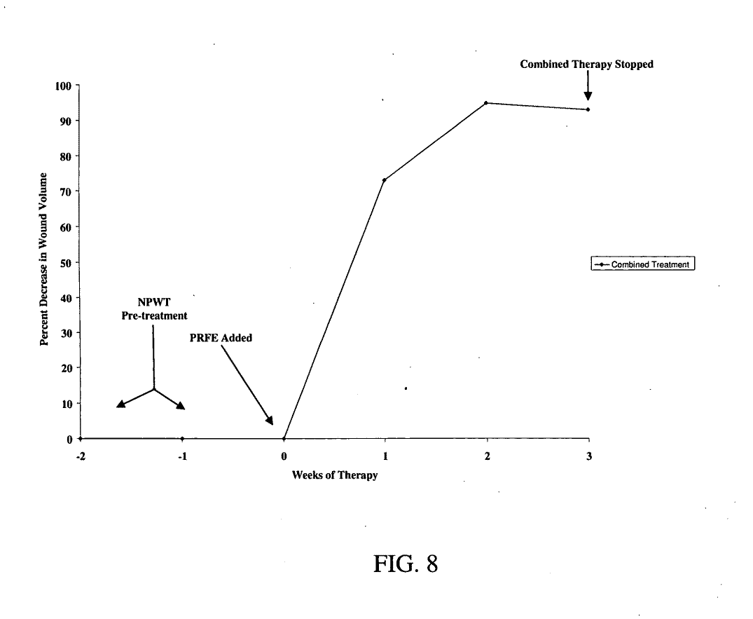

After one week of the combined NPWT and PRFE treatment, the wound had decreased in volume from by 72.5% (FIG. 7 and FIG. 8), and had very healthy granular bed for the first time (FIG. 6B). As shown in TABLE 2 and FIG. 7, after two weeks of the treatment the wound volume had decreased by 95%. At week three of the combined treatment, granulation tissue had grown into the NPWT foam component. Removal of the NPWT foam dressing was traumatic and resulted in an increase in wound volume to 6 cm³. Given that by week three of the combined treatment the wound had decreased over 90% in volume, the NPWT treatment was discontinued (TABLE 2 and FIG. 7).

TABLE 2

| Week of | Wound | Percent | |
|---|---|---|---|
| Treatment | Volume | Decrease | Therapies Used |
| 0 | 80 cm³ | 0 | Initiation of NPWT Alone |
| 1 | 80 cm³ | 0 | NPWT Alone |
| 2 | 80 cm³ | 0 | PRFE Added to NPWT |
| 3 | 22 cm³ | 73% | Combined Treatment |
| 4 | 4 cm³ | 95% | Combined Treatment |
| 5 | 6 cm³ | 93% | PRFE alone |
| 7 | 4 cm³ | 95% | PRFE alone |
| 9 | 0 cm³ | 100% | Wound healed |

Thereafter, wound care consisted of cleansing and plain-gauze packing every two hours and PRFE treatment twice daily. Within two weeks, the wound was nearly closed (FIG. 8) and PRFE treatment was discontinued. Simple dressings were utilized until final closure several days later. The total treatment time with PRFE was 42 days.

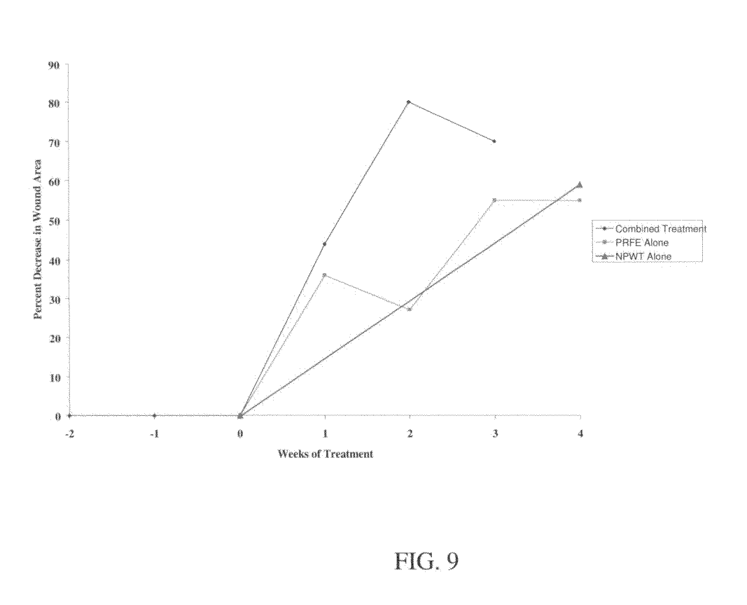

The results of the combined treatment for treating the PD cyst wound were compared to a prospective, open-label, non-comparative case series of 26 patients treated for venous stasis ulcers with PRFE treatment alone²⁸. The mean reduction in wound area over the course of four weeks of PRFE treatment alone was compared to the reduction in wound area using the combined treatment for the PD cyst case study (FIG. 9). The mean decrease in wound area for the venous stasis ulcers treated with PRFE plateaued at about 55%, while the combined treatment achieved an 80% decrease in wound area (FIG. 9). These results suggest that the combined NPWT and PRFE treatment gives an enhanced rate of wound healing compared to PRFE treatment alone.

The results of the combined treatment were also compared to a multicenter, randomized controlled trial of diabetic foot ulcer treatment with NPWT treatment alone²⁹. The mean reduction in wound area at four weeks of treatment with NPWT treatment alone was compared to the reduction in wound volume using the combined treatment for the PD cyst case study (FIG. 9). After four weeks of treatment the NPWT alone treatment gives a decrease in wound area of about 60%, while the combined treatment yielded an 80% decrease in wound area after three weeks (FIG. 9). These results suggest that the combined NPWT and PRFE treatment yields an enhanced rate of wound healing compared to NPWT treatment alone.

### Discussion

Twenty years ago there was little knowledge of the cellular, molecular and physiologic processes involved in dermal wound healing¹⁸. Modern techniques in cellular and molecular biology have revealed the role of many agents including fibroblasts, neutrophils, macrophages, matrix proteins, growth factors, MMPs, TIMPs, ILs, and TNFs¹⁹⁻²¹. Healing of dermal wounds requires coordination of these cellular and biochemical agents through the carefully orchestrated expression of a large set of genes and their products.

Modern wound care protocols have developed from this extensive body of research. Many therapeutics are available to the clinician, including topical, pharmaceutical, biological, antimicrobial, mechanical, and biophysical modalities. Successful healing of complex wounds (such as found in this individual) requires an understanding of wound physiology and the mechanism of action of the various available therapies. NPWT treatment is thought to promote wound healing by removing excess interstitial fluid, decreasing bacterial colonization, and stimulating granulation tissue formation through micromechanical deformation. Interestingly, NPWT treatment alone did not facilitate the closure of this dehisced wound following surgical revision. With the addition of PRFE treatment, rapid acceleration in healing occurred and the wound progressed to closure (FIG. 7 and FIG. 8).

PRFE appears to endogenously stimulate growth factor production and incite mitosis in the wound bed. For example in one in vitro study, George et al. treated human and rat primary fibroblasts and epithelial cells with PRFE for various time periods and at various doses, with cellular proliferation assessed quantitatively by direct counting and spectrophotometric analysis 24 hours after treatment¹⁶. Results were compared with serum-treated controls. The investigators found significantly increased proliferation versus control after one 30 minute PRFE treatment (p<0.001). Further, their results indicated that PRFE treatment induces growth factor production and stimulates cell replication through a calcium-mediated intracellular pathway. That pathway is also known to mediate cell replication, transcription, and programmed cell death and may be the signaling mechanism for the proliferative effect²²⁻²⁴. In another in vitro study, Gilbert et al. reported that cell proliferation in human fibroblasts increased by up to two-fold within 24 hours of treatment using PRFE treatment compared with sham treated controls¹⁷. The authors attributed cell proliferation to the activation by PRFE of the p 44/42 mitogen-activating protein (MAP) kinase pathway. PRFE has been shown to induce proliferation in cultured human dermal fibroblasts and epithelial cells in a dose- and time-dependent fashion²⁵. The effect has also been observed in lymphocytes¹⁶.

Recent reports have cited the effectiveness of PRFE in the treatment of diabetic foot ulcers and sacral pressure ulcers^{26,27}. Pilonidal cyst repair often involves wide excision and healing by secondary intention. As in this case, disruption and complicated recovery are not uncommon. The results in this case suggest that PRFE treatment may help accelerate the healing of complex pilonidal cyst wounds.

### Summary

Pilonidal cyst disease is a significant cause of morbidity among young servicemen. Prolonged wound healing following excision can delay redeployment and impact personnel cost and training efficiency. While NPWT treatment can facilitate wound healing in PN disease, it is not uniformly effective. PRFE treatment, when added to the stalled NPWT treatment wound care regimen in this case, reduced wound volume by 95% in two weeks (FIG. 6 and TABLE 2). With three additional weeks of primary PRFE treatment, the wound progressed to closure. These findings suggest that PRFE treatment may work synergistically with NPWT treatment and may be effective as primary treatment in the treatment of complex PN disease.

The combined NPWT and PRFE treatment was found to enhance the rate of wound healing compared to PRFE treatment alone or NPWT treatment alone. After two weeks of combined treatment percent decrease in wound area was 80%, while the percent decrease in the wound after three weeks of PRFE treatment alone was 55% and four weeks of NPWT treatment alone was 59% (FIG. 9 and TABLE 3). Thus the enhanced rate of wound healing achieved by the combined treatment resulted in a wound that was 25% smaller after two weeks than a wound treated with PRFE treatment alone for three weeks. The enhanced rate of wound healing also resulted in a wound that was 21% smaller after two weeks of combined treatment compared to four weeks of NPWT treatment alone.

TABLE 3

| Week of | Percent | Treatment | Percent | Treatment |
|---|---|---|---|---|
| Treatment | Decrease | Used | Decrease | Used |
| 0 | 0 | PRFE | N/A | N/A |
| 1 | 36% | PRFE | N/A | N/A |
| 2 | 27% | PRFE | N/A | N/A |
| 3 | 55% | PRFE | N/A | N/A |
| 4 | 55% | PRFE | 59% | NPWT |

### Example 3

### Treatment of Non-Healing Pressure Ulcer in a Patient with Spinal Cord Injury Using Pulsed Radio Frequency Energy and Negative Pressure Wound Therapy

### Background

Pressure ulcers in patients with spinal cord injury may become chronic and resistant to treatment. Complex treatment regimens and adjuvant treatments often are necessary, and results may still be uncertain.

### Case Report

A 60-year-old African-American man presented with a stage IV right proximal coccygeal ulcer that had been present for almost 12 years. The patient had a spinal cord injury at C4 (ASIA A) with spastic quadriplegia that he sustained in a fall in 1996. He had numerous co-morbid conditions. He developed the ulcer shortly after his spinal cord injury, which intermittently improved. He had been in nursing facilities but was eventually discharged to the care of his family. His first primary care visit at our clinic revealed a 1.8 cm×2.1 cm×1.4 cm (volume=5.3 cm³) stage IV pressure ulcer on the right proximal coccyx (TABLE 4). The family reported that the ulcer had worsened since he was last seen. Wound care was changed from daily wet-dry dressings to alginate dressings.

During hospitalization for elective subtotal colectomy with endoileostomy in November of 2007, the pressure ulcer worsened. Inpatient examination revealed an increase in ulcer size to 2.5×2×2.5 cm (volume=12.5 cm³) with 2.5 cm undermining (11:00-1:00 o'clock). The ulcer, located on the top of the coccygeal crease, was round and had a moist red wound base. Necrotic tissue was not present, but a small amount of serosanguinous exudate and a slightly foul odor were noted, with maceration around the ulcer margins. His wound care was changed to include daily packing with Sorbsan® (uDL Laboratories, Rockford, Ill.) strips followed by gauze. By December of 2007, the wound had not improved and a negative pressure wound therapy device (NPWT), V.A.C.® (KCI Medical, San Antonio, Tex.), was used.

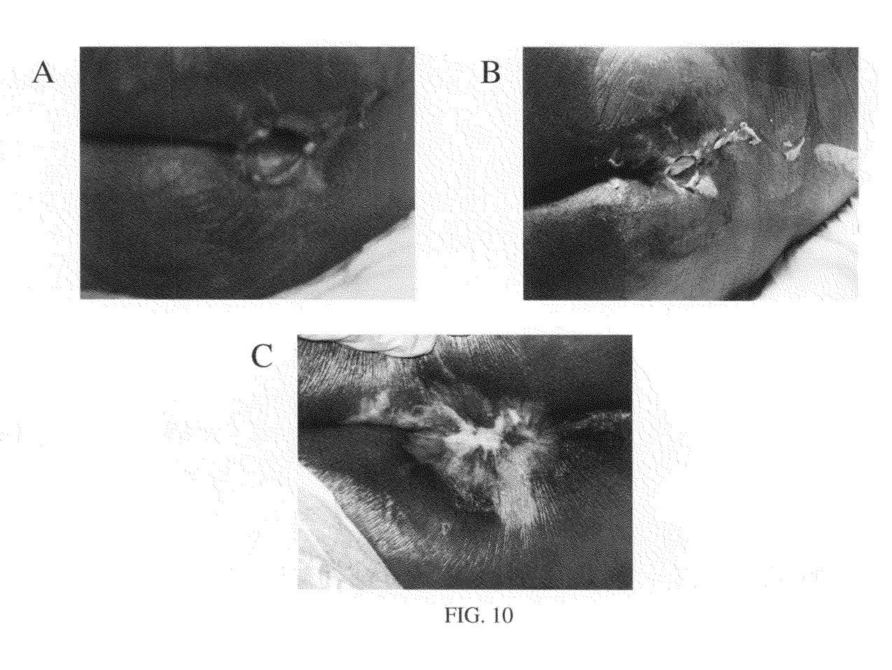

At the initiation of NPWT in December, the ulcer measured 4×1.2×2 cm (volume=9.6 cm³) with 1.8 to 2.5 cm undermining, and after one month it had again enlarged and measured 4.6×1.2×2.4 cm (volume=13.2 cm³) in diameter with 1.3 to 3.8 cm undermining, with a clean dark red crater, foul odor, and a moderate amount of serosanginous exudate (FIG. 10A). Silver antimicrobial dressings were added to the protocol.

Although the wound had decreased in size (2.2×2×0.8; volume=3.5 cm³), the width had almost doubled with 2 cm undermining (9:00-11:O'clock). The patient and family were eager for resolution, as they felt progress was slow.

To optimize treatment and accelerate healing, the decision was made to add a trial of pulsed radio frequency energy treatment (PRFE) (Provant® Therapy System, Regenesis Biomedical, Inc., Scottsdale, Ariz.) along with NWPT (FIG. 10B). PRFE works on a biological level to stimulate dermal proliferation in the wound bed and to induce a cascade of growth factors, cytokines, and extracellular matrix proteins associated with normal wound repair. The patient was treated at home without nursing supervision twice daily for 30 minutes. PRFE therapy requires no dressing change as the energy pulses directly through dressings such as NPWT, casts and compression, preventing transmission of infection to the wound during treatment.

### Results

Two months of combined NPWT and PRFE treatments resulted in a 67% reduction in volume. After 4 months of combined treatment, full closure of the wound was obtained (FIG. 10C).

TABLE 4

| Months of | | | |
|---|---|---|---|
| Treatment | Volume (cm³) | Percent Change | Therapies Used |
| 0 | Occurrence of spinal cord | Wound Development | None |
| | injury | | |
| 0 | 5.3 cm³ without | Initial assessment | None |
| | undermining | | |
| 0 | 12.5 cm³ with undermining | 235% increase after patient | None |
| | of 2.5 cm | hospitalized for surgery | |
| 1 | 9.6 cm³ with undermining | 23% decrease after 1 month. | NPWT Initiated |
| | of 1.8 to 2.5 cm | | |
| 2 | 13.2 cm³ with undermining | 73% increase after 1 month of | NPWT |
| | of 1.3 to 3.5 cm | treatment. | Continued |
| 5 | 3.52 cm³ (width nearly | Progress slow and | PRFE Added |
| | doubled) | fragile peri-wound area. | |
| 7 | “Dime size” | 67% decrease after 2 months of | NPWT Stopped |
| | | combined treatment | |
| 9 | Healed | Closed | PRFE Stopped |

### Discussion

Pressure ulcers are common problems in patients with spinal cord injuries, and many occur quite soon after injury³⁰. When they become chronic stage IV wounds, treatment becomes difficult and healing often is slow, especially if they occur in combination with other co-morbidities such as spinal cord injury, type II diabetes mellitus, coronary artery disease, and anemia of chronic disease. Complex treatment protocols involving multiple advanced wound healing modalities are required for good results. However, even with proper wound care, many ulcers persist, such as in our patient, and further diminish the patient's quality of life (QOL) physically, psychologically, somatically and socially. When PRFE treatment was added to the wound care regimen, the patient's demeanor improved from being combative to congenial.

The traditional therapy of wound bed preparation, debridement, antimicrobial treatment, moisture control, and appropriate dressings is effective and is recommended as treatment for pressure ulcers³¹. It is clear, however, that for chronic, difficult-to-heal ulcers more effective treatment is necessary. Adjuvant treatments, including topical growth factor, NPWT, and electrical stimulation, have been used with variable results³¹. An in vitro study used PRFE to determine its effect on cell proliferation of dermal fibroblast and keratinocyte epithelial cells and found a significant increase in cell proliferation induction or mitosis after one 30-minute treatment⁴. This study and others suggested that PRFE may aid in the healing of wounds^{17,27}. A recent study has also reported wound healing with PRFE in one patient with a long-standing, recalcitrant stage IV sacral ulcer²⁷.

### Conclusions

Combined NPWT and PREF therapy was used in a patient with a stage IV ulcer that had persisted for 12 years. The multi-modality approach resulted in a dramatic reduction in wound size, with near closure after 6 months of treatment, suggesting that PRFE may be of benefit to other patients with spinal cord injury (TABLE 4).

### Example 4

### Treatment of Achilles Tendon Rupture Using Pulsed Radio Frequency Energy and Negative Pressure Wound Therapy

### Introduction

Wounds involving exposed tendon are categorically difficult to manage and slow to heal. Acute Achilles tendon ruptures typically affect men in the third and fourth decades of life, most commonly those participating in physically demanding work, sports, or strenuous recreational activities, and the left Achilles tendon is ruptured more frequently than the right³². Traditional treatment of acute Achilles tendon ruptures can be broadly classified as operative (open or percutaneous) or nonoperative (cast immobilization or functional bracing). Generally, open operative treatment has been used for athletes and young, fit patients; percutaneous operative treatment has been used for those who do not wish to have an open repair (e.g., for cosmetic reasons); and nonoperative treatment has been used for the elderly^{33,34,35,36}. Complications of surgery for Achilles tendon ruptures can include infection, adhesions, and disturbed skin sensibility^{37,38.}

Successful modern treatment of these types of difficult-to-heal wounds demands aggressive, comprehensive, combination therapies to expedite granulation, contraction, and epithelialization. This example describes the use of a pulsed radio frequency energy treatment (PRFE)^{4,5} in conjunction with negative pressure wound therapy (NPWT) in the treatment of an Achilles tendon rupture wound.

### Case History and Results

An active 49-year-old male presented with an Achilles tendon rupture wound on his posterior foot that had persisted for about 3 weeks. The wound had become necrotic. The patient had seen a number of physicians, and none of them had offered him a very good prognosis. He was told that he may need to have surgery, he may need a brace, and that may no longer be able to engage in many of the physical activities to which he was accustomed.

When the patient presented with the Achilles tendon rupture wound, he had been on clindamycin for 3 weeks. The area of erythema around the wound was 7 cm×6 cm with a necrotic tendon and no granulation tissue.

On Day 3, the wound measured 47 mm×45 mm×1 mm (volume=2115 mm³) and the patient was taking hydrocodone 7.5 mg for pain. After 1 week, his antibiotic was switched to sulfamethoxazole and trimethoprim (Bactrim® DS).

On Day 12, treatment with a papain/urea enzymatic debrider (Accuzyme®, Healthpoint Medical) was initiated, and a cadexomer matrix dressing with iodine (Iodosorb®, Smith & Nephew) was applied. At this point, the wound was highly necrotic with some eschar over it, which was scored with a scalpel to allow the enzymatic debriding agent to penetrate more effectively.

On Day 17, the wound measured 50 mm×24 mm×1 mm (volume=1200 mm³) and had a very dry, black eschar. The treatment protocol was modified to an autolytic-enzymatic combination debridment.

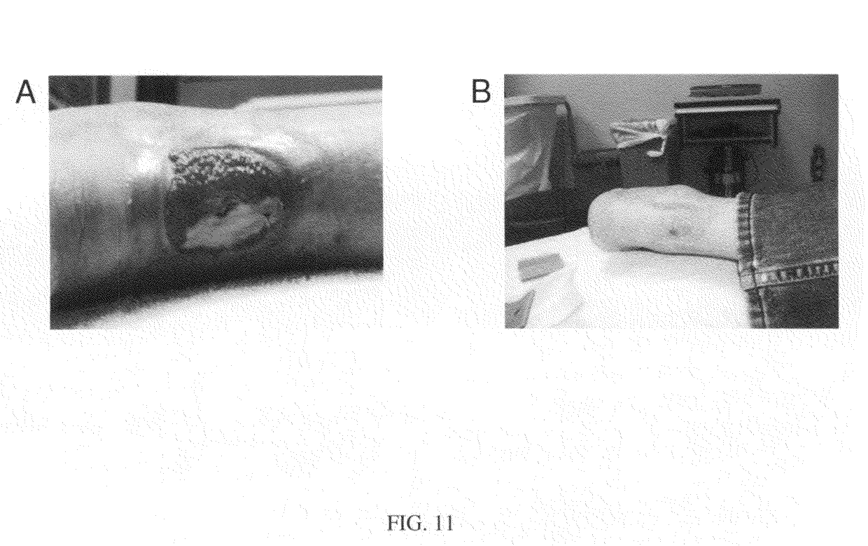

On Day 24, the wound measured 50 mm×24 mm×3 mm (volume=3600 mm³) (FIG. 11A). A sharp debridement was done, and the eschar was removed. On Day 31, another sharp debridment was performed. The tendon was now exposed, but not removed, and it was protected with a gauze dressing.

On Day 32, NPWT (V.A.C.® Therapy, Kinetic Concepts, Inc., San Antonio, Tex.) was started at 125 mm Hg on Mondays, Wednesdays, and Fridays.

On Day 38, a small amount of granulation tissue was seen on the tendon; the wound measured 40 mm×30 mm×3 mm (volume=3600 mm³).

On Day 45, the patient was switched to a different NPWT (Engenex®, ConvaTec and Boehringer Wound Systems, LLC, Norristown, Pa.) at 75 mm Hg. Treatment was continued on Mondays, Wednesdays, and Fridays.

On Day 52, NPWT was continued, the tendon remained moist, granulation buds were seen forming toward the tendon at the base of the wound, and the wound measured 37 mm×27 mm×2 mm (volume=1998 mm³).

On Day 59, the patient was referred to an orthopedic surgeon. On Day 61, he went to see the orthopedic surgeon and was informed that the tendon would need to be removed and that he would have to wear a brace for life. The wound measured 35 mm×25 mm×1 mm (volume=875 mm³) on Day 61. Patient opted to try to heal the wound without surgical intervention.

On Day 63, PRFE treatment (Provant® Therapy System, Regenesis Biomedical Inc., Scottsdale Ariz.) was prescribed as an alternative to excision of the exposed tendon for reduction of pain and edema following surgical debridement of the infected wound.

On Day 66, after 6 PRFE treatments, the tendon was covered at the very top and the very bottom; 10 mm of granulation tissue was observed at the base of the wound. The wound had decreased in total volume by 76% since the start of NPWT therapy.

On Day 73, there was more granulation tissue at the base of the wound, and the tendon was covered at the top and bottom. At the lowest part of the base of the wound, 13 mm of new granulation tissue was observed. On Day 80, granulation tissue was covering the lowest part of the base had increased to 15 mm. There was new epithelium at the edges of the wound. On Day 87, after 3 weeks of PRFE therapy, the wound measured 32 mm×22 mm×1 mm (volume=704 mm³).

On Day 123, the wound had been reduced to 3 pinpoint open areas, and on Day 144, the wound closed (FIG. 11B).

### Conclusion

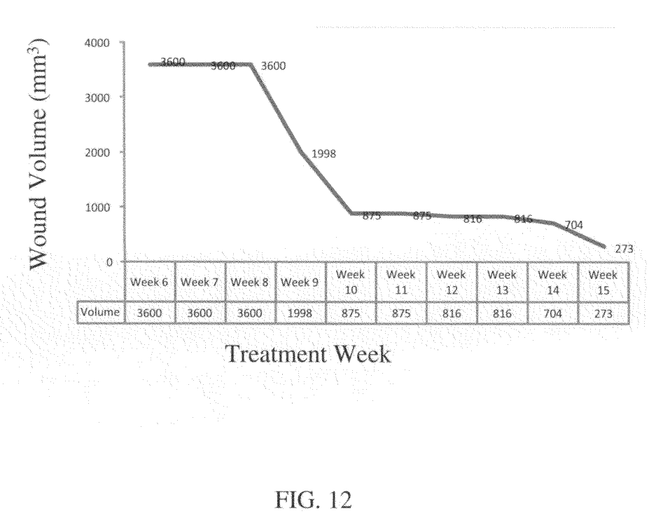

PRFE therapy, in conjunction with NPWT, was found to accelerate the rate of wound closure in this patient with an Achilles tendon rupture (TABLE 5 and FIG. 12). Despite the exposed tendon, the wound granulated and closed rapidly. Surgery was avoided, the patient was able to continue to engage in his active lifestyle, and he has maintained his job as a landscape architect.

TABLE 5

| Day of | Wound | Percent | Treatment |
|---|---|---|---|
| Treatment | Volume (mm³) | Decrease | Used |
| 3 | 2115 mm³ | 0% | None |
| 17 | 1200 mm³ | 43% | None |
| 24 | 3600 mm³ | Increase of 67% | None |
| 32 | 3600 mm³ | 0% | Initiation of |
| | | | NPWT |
| 38 | 3600 mm³ | 0% | NPWT Alone |
| 45 | 3600 mm³ | 0% | NPWT Alone |
| 52 | 1998 mm³ | 44% | NPWT Alone |
| 61 | 875 mm³ | 56% | NPWT Alone |
| 63 | 875 mm³ | 0% | Addition of PRFE |
| | | | to NPWT |
| 66 | 875 mm³ | 0% | PRFE + NPWT |
| 73 | 816 mm³ | 1% | PRFE + NPWT |
| 80 | 816 mm³ | 0% | PRFE + NPWT |
| 87 | 704 mm³ | 14% | PRFE + NPWT |
| 96 | 273 mm³ | 61% | PRFE + NPWT |
| 103 | 273 mm³ | 0% | PRFE + NPWT |
| 111 | 70 mm³ | 74% | PRFE + NPWT |
| 115 | 15 mm³ | 78% | PRFE + NPWT |
| 123 | | N/A | PRFE + NPWT |
| 129 | 4 mm³ | 73% | PRFE + NPWT |
| 144 | 0 mm³ | | PRFE + NPWT |

### SUMMARY

Wounds involving exposed tendon are categorically difficult to manage and slow to heal. While NPWT can facilitate wound healing, it is not uniformly effective. PRFE, when added to the stalled NPWT wound care regimen in this example, rapidly reduced wound volume and the wounds progressed to closure (FIG. 12). These results suggest that PRFE can work synergistically with NPWT and may be effective as primary therapy. Also, patient ccompliance is generally high because PRFE treatments are easy to administer, take only two 30-minute periods per day, and are painless.

### Example 5

### Acceleration of Wound Healing in the Diabetic Mouse Model (db/db) Using Pulse Radio Frequency Energy Treatment and Negative Pressure Wound Therapy

### Introduction

Accelerated rehabilitation of traumatic, surgical and chronic wounds can reduce hospitalization and forestall serious and long lasting complications such as infection, loss of function, loss of limb, and reduced quality of life. Novel and effective wound care modalities are needed which can accelerate wound repair and regeneration. Most needed are modalities which are non-invasive and inexpensive. One such technology is pulsed radio frequency energy treatments (PRFE). Another treatment modality that has been established is negative pressure wound therapy (NPWT).

The application of PRFE to wound repair and regeneration has been hampered by a limited understanding of the biophysical and biological mechanism(s) of action. The interaction and interplay of electromagnetic frequency, pulse width, and wave form on various tissues and organ systems needs to be examined using informative in vitro and in vivo models. In a previous study, the diabetic db/db mouse model was established to be an informative experimental model for determining the effects of PRFE on in vivo animal wounds.

The objective of this study is to determine the effect on wound healing using the diabetic db/db mouse model when both PRFE and negative pressure wound therapy (NPWT) are used to treat full thickness dermal wounds. Another objective is to determine if PRFE and NPWT can act additively or synergistically to increase the rate of wound closure.

### Methods and Results

The PRFE and NPWT treatments will be performed using three Provant® 4201 “active units” (Provant® Therapy System), three Provant® 4201 “sham units” (Provant® Therapy System), and 8 mouse NPWT treatment chambers

The study will use diabetic db/db mice as the animal model.

There will be 5 experimental groups consisting of 10 db/db mice each. The treatment groups will consist of:
1. Control (sham NPWT and/or sham PRFE)
2. NPWT
3. PRFE
4. NPWT+PRFE in Parallel
5. NPWT (7 days) followed by PRFE

Treatment will be blinded. Single one square centimeter excisional wounds will be generated on the dorsum of each db/db mouse using standard procedures. Wounds will be photographed on a twice-weekly basis and the areas of each remaining open wound will be recorded. If there are any deaths or infections, the schedule will be slightly adjusted.

The experiment will progress until wound closure or for a set time, depending information requirements.

Mice will be sacrificed for histological assessment according to the following schedule: 1 mouse of day 7, 3 mice on day 14, 3 mice on day 21, and 3 mice when wound is all healed.

Wound area, epithelial area, and open wound area measurements will be plotted as a function of time. Detailed quantitative histological analysis including thickness of regenerating epidermis and granulating dermis will be performed. Statistical comparisons between groups will be performed using ANOVA or t-tests, as applicable.

Histological samples are stained with H&E for morphological studies. Immunohistochemical stains will use Ki-67 for general cellular proliferation studies and CD-31 for endothelial cell activities. Real time RT-PCR will also be performed on samples.

### REFERENCES

1. Ozkan O et al. Rationale for reconstruction of large scalp defects using the anterolateral thigh flap: structural and aesthetic outcomes, J Reconstr Microsurg. 21(8):539-45 (2005).
2. Pitanen J M et al. Immediate coverage of exposed, denuded cranial bone with split-thickness skin grafts, Ann Plast Surg. 45(2):118-21 (2000).
3. Becker G D et al. Secondary intention healing of exposed scalp and forehead bone after Mohs surgery, Otolaryngol Head Neck Surg. Dec:121(6):751-4 (1999).
4. George F R et al. In vitro mechanisms of Cell Proliferation Induction: a novel bioactive treatment for accelerating wound healing, Wounds, 14(3):107-115 (2002).
5. Gilbert L et al. The Provant Wound Closure System Induces Activation of p 44/42 MAP Kinase in Normal Cultured Human Fibroblasts, Ann. NY Acad Sci. 961:168-171.
6. Al-Khayat H Al-Khayat H, Risk factors for wound complication in pilonidal sinus procedures, J Am Coll Surg. 205: 3; 439-44. Epub 2007 Jun. 27.
7. da Silva J H, Pilonidal cyst: cause and treatment, Dis Colon Rectum 2000 August; 43(8):1146-56.
8. Buie L A, Jeep disease, Southern Med J, (1944) 37:103-0.
9. Bascome J, Pilonidal sinus: experience with Karydakis flap, Br J Surg, (1998) 85-874.
10. Abramson O J: Outpatient management of pilonidal sinuses: excision and primary closure, Mil Med 1978; 143:753-757.
11. Stephens F O, Sloan D R: Conservative management of pilonidal sinus, Surg Gynecol Obstet 1969; 129:786.
12. Allen-Mersh G: Pilonidal sinus: finding the right track for treatment, Br J Surg, 1990; 77: 123132.
13. C Perruchoud, H Vuilleumier, J C Givel, Pilonidal sinus: how to choose between excision and open granulation versus excision and primary closure? Swiss Surg. 2002; 8 (6): 255-8 12520844.
14. Bendewald F P, Cima R R, Metcalf D R, Hassan I, Using negative pressure wound therapy following surgery for complex pilonidal disease: a case series, Ostomy Wound Manage. 2007 May; 53(5):40-6.
15. Lynch J B, Laing A J, Regan P J, Vacuum-assisted closure therapy: a new treatment option for recurrent pilonidal sinus disease, Report of 3 cases, Dis Colon Rectum. 2004 June; 47(6):929-32.
16. George F R, Lukas R J, Moffett J, Ritz M C. In-vitro mechanisms of cell proliferation induction: a novel bioactive treatment for accelerating wound healing, Wounds: Comp Clin Res Pract 2002.14:10.
17. Gilbert T L, Griffin N, Moffett J, Ritz M C, George F R. The provant wound closure system induces activation of p 44/42 MAP kinase in normal cultured human fibroblasts, Ann N Y Acad Sci. 2002; 961: 168-171.
18. Waldorf H, Fewkes J, Wound healing, Adv Dermato 1995. 10:77-96.
19. Grant, G. A., A. Z. Eisen, B. L. Manner, W. T. Roswit, and G. I. Goldberg, The activation of human skin fibroblast procollagenase. Sequence identification of the major conversion products, J. Biol. Chem. 1987; 262:5886-5889.
20. A Hascall V C and Hascall G K. 1981. Proteoglycans In Cell Biology of Extracellular Matrix, Hay E B, edito10.
21. Gillitzer, R. and M. Goebeler. Chemokines in cutaneous wound healing, J. Leukoc. Biol. 2001; 69:513-521.
22. Davis R J. Transcriptional regulation by MAP kinases, Mol Reprod Dev. 1995; 42: 459-467.
23. Gilbert T L, Griffin N, Moffett J, Ritz M C, George F R. The provant wound closure system induces activation of p 44/42 MAP kinase in normal cultured human fibroblasts, Ann N Y Acad Sci. 2002; 961: 168-171.
24. Dent P, Jarvis W D, Birrer M J, Fisher B, Schmidt-Ullrich R K, Grant S. The roles of signaling by the p 42/p 44 mitogen-activated protein (MAP) kinase pathway; a potential route to radio- and chemosensitization of tumor cells resulting in the induction of apoptosis and loss of clonogenicity, Leukemia. 1998; 12: 1843-1850.
25. Regenesis Biomedical. Operating Manual: Provant Wound Therapy System, 2051-011, Scottsdale, Ariz.: Regenesis Biomedical, Inc; 2007.
26. Larsen J A, Overstrert J. Pulsed Radio Frequency Energy in the Treatment of Complex Diabetic Foot Wounds Two Cases, JWOCN 2008; 35(5): 523-527.
27. Porreca E G, Giodano-Jablon G M. Treatment of Severe (Stage III and IV) Chronic Pressure Ulcers using Pulsed radio Frequency Energy in a Quadraplegic Patient. ePlasty, The online Journal of Plastic Surgery. 2008; 8: 451-454.
28. Isenberg R A. Cell Proliferation Induction (CPI®) Wound Therapy in Venous Stasis Ulcers: A Prospective, Multi-Center Case Series of 26 Ulcers Across Various Sites of Care. J WOCN 2008. 35(3S): S64.
29. Blume P A, Walters J, Payne W et al. Comparison of Negative Pressure Wound Therapy Using Vacuum-Assisted Closure with Advanced Moist Wound Therapy in the Treatment of Diabetic Foot Ulcers: a multicenter randomized controlled trial. Diabetes Care 2008; 31:631-636.
30. Mawson A R, Biundo J J, Neville P, et al. Risk factors for Early-Occuiring Pressure Ulcers Following Spinal Cord Injury. Am J Phys Med Rehabil 1988; 67:123-127.
31. Whitney J, Phillips L, Aslam R, Barbul A, Gottrup F, et al. Guidelines for the Treatment of Pressure Ulcers. Wound Rep Reg. 2006; 14: 663-679.
32. Khan R J, Fick D, Keogh A, Crawford J, Brammar T, Parker M. Treatment of Acute Achilles Tendon Ruptures. A Meta-Analysis of Randomized, Controlled Trials. J Bone Joint Surg Am. 2005; 87 (10): 2202-2210.
33. Bossley C J. Rupture of the Achilles Tendon. J Bone Joint Surg Am. 2000; 82-A (12): 1804.
34. Maffulli N. Rupture of the Achilles Tendon. J Bone Joint Surg Am. 1999; 81(7): 1019-1036.
35. Raisbeck C C. Rupture of the Achilles Tendon. J Bone Joint Surg Am. 2000; 82-A (12): 1804-1805.
36. Popovic N, Lemaire R. Diagnosis and Treatment of Acute Ruptures of the Achilles Tendon. Current Concepts Review. Acta Orthop Belg. 1999; 65(4): 458-471.
37. Khan R J, Fick D, Brammar T J, Crawford J, Parker M J. Interventions for Treating Acute Achilles Tendon Ruptures. Cochrane Database Syst Rev. 2004;(3): CD003674
38. Paavola M, Orava S, Leppilahti J, Kannus P, Järvinen M. Chronic Achilles Tendon Overuse Injury: Complications After Surgical Treatment. An Analysis of 432 Consecutive Patients. Am J Sports Med. 2000; 28(1): 77-82.

What is claimed is:

1. A method for treating a wound of an individual and for enhancing a rate of wound healing, the method comprising:
applying, for a first period of time, a negative pressure treatment to the wound without applying a pulsed radio frequency treatment; and
applying, for a second period of time subsequent to the first period time, a pulsed radio frequency energy treatment to the wound, while maintaining the negative pressure treatment to enhance the rate of wound healing,
wherein the negative pressure treatment and the pulsed radio frequency energy treatment are applied concurrently for the duration of the second period of time.

2. The method of claim 1, wherein the negative pressure treatment is applied continuously throughout the first period of time.

3. The method of claim 1, wherein the negative pressure treatment is applied intermittently throughout the first period of time.

4. The method of claim 1, wherein the negative pressure treatment is applied 24-hours a day throughout the first period of time.

5. The method of claim 1, wherein the negative pressure treatment is applied for less than 24-hours a day throughout the first period of time.

6. The method of claim 1, wherein the enhanced rate of wound healing is greater than a rate of wound healing achieved by the negative pressure treatment alone or the pulsed radio frequency energy treatment alone.

7. The method of claim 1, wherein the second period of time is a period of time sufficient to achieve at least a 90% decrease in wound volume.

8. The method of claim 1, wherein the wound is selected from the group consisting of a chronic wound, a large, deep, open wound, a graft and flap site wound, a full thickness burn, a partial thickness burn, a diabetic ulcer, a pressure ulcers, a decubitus ulcer; an arterial ulcer; an avulsion injury a pilonidal disease, a cysts, an acute wound, a tendon rupture wound, a postoperative incision, a postoperative wound, a traumatic wound, a dermatology condition, scleroderma, atrophy blanche disease, trauma, a bomb blast or other military-type inflicted wound, a gunshot wound, a bite, and a wound dehiscence.

9. The method of claim 1, wherein the negative pressure treatment results in an effect selected from the group consisting of: removing excess interstitial fluid, decreasing bacterial colonization, and stimulating granulation tissue formation.

10. The method of claim 1, wherein the pulsed radio frequency energy treatment results in an effect selected from the group consisting of: stimulating growth factor production and stimulating cell proliferation.

11. A method for treating a wound of an individual and for enhancing a rate of wound healing, the method comprising:
applying concurrently a negative pressure treatment and a pulsed radio frequency energy treatment, wherein the negative pressure treatment and pulsed radio frequency energy treatment are maintained for a period of time sufficient to achieve the enhanced rate of wound healing,
wherein the enhanced rate of wound healing results in at least a 90% decrease in wound volume.

12. The method of claim 11, wherein the negative pressure treatment is applied continuously.

13. The method of claim 11, wherein the negative pressure treatment is applied intermittently.

14. The method of claim 11, wherein the negative pressure treatment is applied 24-hours a day throughout the period of time.

15. The method of claim 11, wherein the negative pressure treatment is applied for less than 24-hours a day throughout the period of time.

16. The method of claim 11, wherein the enhanced rate of wound healing is greater than a rate of wound healing achieved by the negative pressure treatment alone or the pulsed radio frequency energy treatment alone.

17. The method of claim 11, wherein the wound is selected from the group consisting of a chronic wound, a large, deep, open wound, a graft and flap site wound, a full thickness burn, a partial thickness burn, a diabetic ulcer, a pressure ulcers, a decubitus ulcer; an arterial ulcer; an avulsion injury a pilonidal disease, a cysts, an acute wound, a tendon rupture wound, a postoperative incision, a postoperative wound, a traumatic wound, a dermatology condition, scleroderma, atrophy blanche disease, trauma, a bomb blast or other military-type inflicted wound, a gunshot wound, a bite, and a wound dehiscence.

18. The method of claim 11, wherein the negative pressure treatment results in an effect selected from the group consisting of: removing excess interstitial fluid, decreasing bacterial colonization, and stimulating granulation tissue formation.

19. The method of claim 11, wherein the pulsed radio frequency energy treatment results in an effect selected from the group consisting of: stimulating growth factor production and stimulating cell proliferation.
